(19) United States
(12) Patent Application Publication
(10) Pub. No.: US 2020/0353020 A1
(43) Pub. Date: Nov. 12, 2020

(54) COMPOSITION FOR PROMOTING HYALURONIC ACID PRODUCTION

(71) Applicant: House Wellness Foods Corporation, Hyogo (JP)

(72) Inventors: Hiroko Nakai, Hyogo (JP); Yoshitaka Hirose, Hyogo (JP); Shinji Murosaki, Hyogo (JP)

(21) Appl. No.: 16/640,501

(22) PCT Filed: Aug. 31, 2018
(86) PCT No.: PCT/JP2018/032316

§ 371 Date: Feb. 20, 2020

(30) Foreign Application Priority Data
Sep. 1, 2017 (JP) 2017-168930

(51) Int. Cl. A61K 35/747; A23L 33/135; A61P 19/02; A61P 17/00

(52) U.S. Cl. CPC A61K 35/747; A23L 33/135; A23L 33/30; A61P 17/00; A61P 19/02

(57) ABSTRACT

An object of the present invention is to provide a composition for promoting fibroblast proliferation and/or a composition for promoting hyaluronic acid synthase gene expression. The present invention provides a composition for promoting fibroblast proliferation and/or a composition for promoting hyaluronic acid synthase gene expression, the compositions each comprising Lactobacillus plantarum L-137.

### TECHNICAL FIELD

The present invention relates to a composition for promoting hyaluronic acid production. In particular, the present invention relates to a composition for promoting hyaluronic acid production, the composition comprising Lactobacillus plantarum L-137.

### BACKGROUND ART

Lactobacillus plantarum L-137 is known to have various functions, including the ability to prevent a cold (Patent Literature 1 to 6 and Non-patent Literature 1 to 10). However, the ability of Lactobacillus plantarum L-137 to promote fibroblast proliferation and hyaluronic acid synthase gene expression has not been reported yet or not even suggested.

### CITATION LIST

### Patent Literature

Patent Literature 1: JP 2010-95465 A
Patent Literature 2: JP H10-167972 A
Patent Literature 3: WO 2014/199448
Patent Literature 4: WO 2008/018143
Patent Literature 5: WO 2004/084923
Patent Literature 6: WO 2004/084922

### Non-Patent Literature

Non-patent Literature 1: Iwasaki et al., Oral Health Prev Dent., 2016; 14(3):207-14.
Non-patent Literature 2: Hatano et al., Int Immunopharmacol., 2015; April; 25(2):321-31.
Non-patent Literature 3: Hirose et al., J Nutr Sci., 2013 Dec. 6; 2:e39.
Non-patent Literature 4: Fujiki et al., Biosci Biotechnol Biochem., 2012; 76(5):918-22.
Non-patent Literature 5: Arimori et al., Immunopharmacol Immunotoxicol. 2012 December; 34(6):937-43.
Non-patent Literature 6: Hirose et al., Microbiol Immunol. 2010 March; 54(3):143-51.
Non-patent Literature 7: Maeda et al., Int Immunopharmacol. 2009 August; 9(9):1122-5.
Non-patent Literature 8: Hirose et al., J Nutr. 2006 December; 136(12):3069-73.
Non-patent Literature 9: Murosaki et al., Cancer Immunol Immunother. 2000 June; 49(3):157-64.
Non-patent Literature 10: Murosaki et al., J Allergy Clin Immunol. 1998 July; 102(1):57-64.

### SUMMARY OF INVENTION

### Technical Problem

An object of the present invention is to provide a composition for promoting fibroblast proliferation and/or a composition for promoting hyaluronic acid synthase gene expression.

### Solution to Problem

The inventors found that a composition containing Lactobacillus plantarum L-137 has an effect of promoting fibroblast proliferation and/or an effect of promoting hyaluronic acid synthase gene expression. The inventors made further study and completed the present invention.

That is, the present invention relates to the following.

[1] A composition for promoting fibroblast proliferation, the composition comprising Lactobacillus plantarum L-137.
[2] A composition for promoting hyaluronic acid synthase gene expression, the composition comprising Lactobacillus plantarum L-137.
[3] The composition according to the above [1] or [2] for use in the promotion of hyaluronic acid production.
[4] The composition according to the above [1] or [2] for use in the prevention, amelioration or treatment of a skin disease or arthritis.
[5] A method for producing a composition comprising hyaluronic acid, the method comprising allowing (1) (a) Lactobacillus plantarum L-137 and (b) an immune cell or a substance produced by an immune cell to contact with (2) fibroblasts.
[6] A hyaluronic acid-containing composition produced by the production method according to the above [5].
[7] The composition according to any one of the above [1] to [4] and [6], which is a food or drink product.
[8] The composition according to the above [7], wherein the food or drink product is a food additive or a dietary supplement.
[9] A method for promoting fibroblast proliferation and/or hyaluronicacid synthase gene expression, the method comprising administering an effective amount of Lactobacillus plantarum L-137 to a subject.
[9-2] The method according to the above [9], wherein the Lactobacillus plantarum L-137 promotes hyaluronic acid production.
[9-3] The method according to the above [9] or [9-2], wherein the Lactobacillus plantarum L-137 prevents, ameliorates or treats a skin disease or arthritis.
[9-4] The method according to any one of the above [9] to [9-3], wherein the Lactobacillus plantarum L-137 is contained in a food or drink product.
[9-5] The method according to the above [9-4], wherein the food or drink product is a food additive or a dietary supplement.
[10] Lactobacillus plantarum L-137 for use in the promotion of fibroblast proliferation and/or hyaluronic acid synthase gene expression.
[10-2] The Lactobacillus plantarum L-137 for use according to the above [10], wherein the Lactobacillus plantarum L-137 promotes hyaluronic acid production.
[10-3] The Lactobacillus plantarum L-137 for use according to the above [10] or [10-2], wherein the Lactobacillus plantarum L-137 prevents, ameliorates or treats a skin disease or arthritis.
[10-4] The Lactobacillus plantarum L-137 for use according to any one of the above [10] to [10-3], wherein the Lactobacillus plantarum L-137 is contained in a food or drink product.
[10-5] The Lactobacillus plantarum L-137 for use according to the above [10-4], wherein the food or drink product is a food additive or a dietary supplement.
[11] Use of Lactobacillus plantarum L-137 for the production of a medicament for promoting fibroblast proliferation and/or hyaluronic acid synthase gene expression.
[11-2] The use according to the above [11], wherein the Lactobacillus plantarum L-137 promotes hyaluronic acid production.
[11-3] The use according to the above [11] or [11-2], wherein the Lactobacillus plantarum L-137 prevents, ameliorates or treats a skin disease or arthritis.
[11-4] The use according to any one of the above [11] to [11-3], wherein the Lactobacillus plantarum L-137 is contained in a food or drink product.
[11-5] The use according to the above [11-4], wherein the food or drink product is a food additive or a dietary supplement.
[12] Use of Lactobacillus plantarum L-137 for the promotion of fibroblast proliferation and/or hyaluronic acid synthase gene expression.
[12-2] The use according to the above [12], wherein the Lactobacillus plantarum L-137 promotes hyaluronic acid production.
[12-3] The use according to the above [12] or [12-2], wherein the Lactobacillus plantarum L-137 prevents, ameliorates or treats a skin disease or arthritis.
[12-4] The use according to any one of the above [12] to [12-3], wherein the Lactobacillus plantarum L-137 is contained in a food or drink product.
[12-5] The use according to the above [12-4], wherein the food or drink product is a food additive or a dietary supplement.

### Advantageous Effects of Invention

The composition according to the present invention preferably has one or more effects selected from the following:

(1) an effect of promoting fibroblast proliferation,
(2) an effect of promoting the expression of hyaluronic acid synthase genes, such as HAS1 and HAS2,
(3) an effect of promoting hyaluronic acid production,
(4) an effect of preventing, ameliorating or treating skin diseases, for example, atopic dermatitis, psoriasis, etc., and
(5) an effect of preventing, ameliorating or treating arthritis, for example, knee pain, cartilage disorder, knee osteoarthritis, cartilage defect, cartilage damage, meniscus injury, etc.

### BRIEF DESCRIPTION OF DRAWINGS

FIG. 1 is a chart showing hyaluronic acid synthase 1 (HAS1) gene expression levels (Student's t-test, *p<0.01 vs. Comparative Example 1, mean±standard deviation).

FIG. 2 is a chart showing hyaluronic acid synthase 2 (HAS2) gene expression levels (Student's t-test, *p<0.01 vs. Comparative Example 1, mean±standard deviation).

FIG. 3 is a chart showing the proliferation activity of fibroblasts (Student's t-test, *p<0.01 vs. Comparative Example 2, mean±standard deviation).

FIG. 4 is a chart showing hyaluronic acid production levels (Student's t-test, *p<0.01 vs. Comparative Example 2, mean±standard deviation).

### DESCRIPTION OF EMBODIMENTS

The present invention provides (1) a composition for promoting fibroblast proliferation, the composition comprising Lactobacillus plantarum L-137, and/or (2) a composition for promoting hyaluronic acid synthase gene expression, the composition comprising Lactobacillus plantarum L-137 (hereinafter, these compositions are also called the composition of the present invention).

The composition of the present invention comprises Lactobacillus plantarum L-137. Lactobacillus plantarum L-137 was deposited with the International Patent Organism Depositary of the Incorporated Administrative Agency National Institute of Technology and Evaluation (address: #120, 2-5-8 Kazusakamatari, Kisarazu-shi, Chiba 292-0818) under Accession No. FERM BP-08607 (transferred from FERM P-15317 deposited on Nov. 30, 1995). Lactobacillus plantarum L-137 herein includes a mutant of Lactobacillus plantarum L-137 that has the same characteristics as those of Lactobacillus plantarum L-137.

The composition of the present invention may contain another ingredient in addition to Lactobacillus plantarum L-137. That is, the composition of the present invention can be produced by combining Lactobacillus plantarum L-137 with, if necessary, another desired ingredient, and stirring the mixture, and performing other necessary procedures.

Such an ingredient other than Lactobacillus plantarum L-137 is not limited as long as the effects of the present invention are not lost, and examples thereof include any ingredients known in the field of medicines, pharmaceuticals, food products, etc. Examples of the ingredient other than Lactobacillus plantarum L-137 include immune cells, substances produced by immune cells, known additives, etc. The composition of the present invention that contains an immune cell and/or a substance produced by an immune cell can more effectively exhibit fibroblast proliferation-promoting activity and/or hyaluronic acid production-promoting activity.

Examples of the immune cells in this disclosure include lymphocytes, such as B cells or T cells, macrophages, etc. These cells can be isolated from the blood, spleen, or other organs of mammals, birds or other animals by a known method, or can be obtained as commercial products. The substance produced by immune cells may be any substance produced by immune cells, but is preferably a substance whose production from immune cells is promoted as a result of contact of the immune cells with Lactobacillus plantarum L-137. Examples of the substance (ingredient) produced by immune cells include, for example, cytokines such as IL-12, IFN-γ and TNF-α, and various growth factors.

The amount of Lactobacillus plantarum L-137 contained in the composition of the present invention is not limited to a particular amount as long as the effects of the present invention are exhibited, and may be, for example, in the range of about 0.001 by mass to 100: by mass, or 1 to 50% by mass, based on 100% by mass of the composition.

The number of the immune cells contained in the composition of the present invention is not limited to a particular number as long as the effects of the present invention are exhibited, and may be 1 to 1×10⁷ cells/mL.

Method for Obtaining Lactobacillus plantarum L-137

Lactobacillus plantarum L-137 may be cultured by a known method, a method known per se, or a modified method thereof. Lactobacillus plantarum L-137 can be obtained by culture in various culture media, such as a natural medium, a synthetic medium, a semi-synthetic medium, etc. The culture medium preferably contains a nitrogen source and/or a carbon source. Examples of the nitrogen source include meat extract, peptone, gluten, casein, yeast extract, amino acids, etc. Examples of the carbon source include glucose, xylose, fructose, inositol, maltose, starch syrup, koji extract, starch, bagasse, wheat bran, molasses, glycerol, etc. These may be used alone or in combination of two or more types.

The culture medium may contain, in addition to the nitrogen source and/or the carbon source, minerals such as ammonium sulfate, potassium phosphate, magnesium chloride, sodium chloride, iron, manganese and molybdenum; vitamins; etc. These may be added alone or in combination of two or more types.

In an embodiment of the present invention, the culture temperature of Lactobacillus plantarum L-137 is, for example, usually about 25 to 40° C., and preferably about 27 to 35° C.

In an embodiment of the present invention, the culture time of Lactobacillus plantarum L-137 is about 12 to 48 hours, optionally with aeration by shaking. In an embodiment of the present invention, Lactobacillus plantarum L-137 may be cultured with aeration by shaking. The pH of the culture medium is not limited to a particular value, but is usually about pH 3 to 6, preferably about pH 4 to 6.

The bacterial cells of Lactobacillus plantarum L-137 may be viable cells or dead cells, but preferred are dead cells due to stability, ease of handling, etc.

The bacterial cells may be collected at the end of culture to prepare heat-killed bacterial cells. Alternatively, the bacterial cells in culture medium may be subjected to heat treatment to prepare heat-killed bacterial cells without separation from the culture medium, and the heat-killed bacterial cells may then be collected from the culture medium. The collection of the bacterial cells from the culture medium is performed by, for example, adding distilled water to the culture medium, removing the supernatant by centrifugation or other means, repeating this procedure if necessary, and collecting the bacterial cells by centrifugation, filtration or other means.

The heat-killed bacterial cells of Lactobacillus plantarum L-137 can also be obtained by subjecting the collected viable cells or the culture medium containing viable cells to heat treatment to inactivate the bacterial cells, followed by drying using an appropriate means, such as spray drying, freeze drying, etc. The heating temperature is usually about 60 to 100° C., preferably about 70 to 90° C. The means for heating may be a known means using a heater. The heating time is usually about 5 to 40 minutes, preferably about 10 to 30 minutes, after the desired temperature is reached.

The killed bacterial cells prepared as described above may further be subjected to grinding, crushing, lyophilization, or other means to prepare a processed product of the killed bacterial cells. Such a processed product of the killed bacterial cells is also suitable for use as the killed bacterial cells.

### Usage

The route of administration of the composition of the present invention is not limited to a particular one, and the composition may be administered orally or parenterally to a mammal etc. When orally administered, the composition of the present invention is allowed to come in contact with immune cells in the body to exert its effects. When parenterally administered, the composition of the present invention is allowed to come in contact with immune cells present in the skin etc. to exert its effects.

The dosage or intake of Lactobacillus plantarum L-137 administered orally or via an injection can be determined depending on the age and body weight of the subject, the symptoms, the administration time, the dosage form, the mode of administration, a medicine to be co-administered, etc. For example, the intake of Lactobacillus plantarum L-137 based on the weight of the dried dead cells is preferably about 0.5 to 200 mg, more preferably about 1 to 100 mg, and further more preferably about 2 to 50 mg per adult human (about 60 kg) per day. The intake of Lactobacillus plantarum L-137 based on the number of the viable cells is preferably about 5×10⁸ to 2×10¹¹ cfu (colony forming unit), more preferably about 1×10⁹ to 1×10¹¹ cfu per adult human (about 60 kg) per day. The frequency of intake may be once a day or multiple times a day.

When administered via external application, the amount of applied Lactobacillus plantarum L-137 may be appropriately selected depending on the area of the skin to be treated. Typically, the amount of applied Lactobacillus plantarum L-137 is preferably about 0.01 to 2.5 mg, more preferably about 0.02 to 1 mg, per day for about 10 cm of the applied site. The daily dose may be administered or applied as a single dose per day or as multiple divided doses per day.

The composition of the present invention, when orally administered, may be in the form of a solid formulation, such as a powder, granules, a pill, a tablet and a capsule, or in the form of a liquid formulation, such as a syrup. In the production process of these dosage forms, a carrier or additive appropriate for such a dosage form can be used. Examples of the carrier or additive include excipients (sodium polyacrylate, calcium polyacrylate, carboxymethylcellulose, lactose, dextrin, cornstarch, crystalline cellulose, saccharose, sodium chloride, glucose, urea, starch, calcium carbonate, kaolin, silicic acid, potassium phosphate, etc.), lubricants (magnesium stearate, sucrose fatty acid ester, glycerol fatty acid ester, purified talc, polyethylene glycol, etc.), disintegrants (carboxymethylcellulose calcium, anhydrous dibasic calcium phosphate, carboxymethylcellulose sodium, low-substituted hydroxypropyl cellulose, dry starch, sodium alginate, agar powder, sodium hydrogen carbonate, calcium carbonate, etc.), binders (hydroxypropyl cellulose, liquid gum arabic, water, ethanol, propanol, simple syrup, glucose in water, starch in water, gelatin in water, carboxymethylcellulose, methylcellulose, polyvinylpyrrolidone, etc.), solubilizers (gum arabic, polysorbate 80, etc.), absorption enhancers (sodium lauryl sulfate etc.), buffering agents (phosphate buffer solution, acetate buffer solution, borate buffer solution, carbonate buffer solution, citrate buffer solution, Tris buffer solution, etc.), preservatives (methyl parahydroxybenzoate, ethyl parahydroxybenzoate, propyl parahydroxybenzoate, butyl parahydroxybenzoate, chlorobutanol, benzyl alcohol, benzalkonium chloride, sodium dehydroacetate, disodium edetate, etc.), thickeners (propylene glycol, glycerol, hydroxyethyl cellulose, hydroxypropyl cellulose, polyvinyl alcohol, polyethylene glycol, etc.), stabilizers (sodium bisulfite, sodium thiosulfate, disodium edetate, sodium citrate, ascorbic acid, dibutylhydroxytoluene, etc.), and PH adjustors (hydrochloric acid, sodium hydroxide, phosphoric acid, acetic acid, etc.). As needed, the dosage forms may be coated with a coating agent (saccharose, gelatin, hydroxypropyl cellulose, hydroxypropyl methylcellulose phthalate, etc.). The coating may have two or more layers.

The type of the composition of the present invention is not limited to a particular one, and may be a food or drink product, a feed, a medicament, a quasi-drug, a cosmetic product, etc. Preferred is a food or drink product etc.

The food or drink product containing the composition of the present invention may further contain a food additive commonly used in a food or drink product. Examples of the food additive include sweeteners, colorants, preservatives, thickeners, antioxidants, color fixatives, bleaching agents, antifungal agents, gum bases, bittering agents, enzymes, brighteners, acidulants, seasonings, emulsifiers, fortifiers, processing aids, flavors, and spice extracts. The food or drink product includes foods with functional claims, foods for specified health use, health foods and foods for the sick.

The form of the food or drink product suitable for the present invention is not limited to a particular one. Specific examples of the form of the food or drink product include food additives and the so-called dietary supplements in the form of a tablet, granules, a powder, or an energy drink. Other examples thereof include drinks such as tea drinks, refreshing drinks, carbonated drinks, nutritional drinks, fruit juice and lactic drinks; noodles such as buckwheat noodles, wheat noodles, Chinese noodles and instant noodles; sweets and bakery products such as drops, candies, gum, chocolate, snack, biscuits, jelly, jam, cream, pastry and bread; fishery or livestock products such as fish sausage, ham and sausage; dairy products such as processed milk and fermented milk; fats, oils and processed foods thereof, such as vegetable oil, oil for deep frying, margarine, mayonnaise, shortening, whipped cream and dressings; seasonings such as sauce and dipping sauce; retort pouch foods such as curry, stew, rice-bowl cuisine, porridge and rice soup; frozen desserts, such as ice cream, sherbet and shaved ice; etc.

The feed containing the composition of the present invention may be a feed for livestock animals, such as cow, horses and pigs; a feed for poultry such as chickens; a feed for farming, such as fish farming; and a feed for pet animals, such as dogs and cats. The feed of the present invention can be produced and processed by adding the composition of the present invention to a feed, or by using a common production method of a feed.

The composition of the present invention may be formulated into a medicament. The medicament can be produced by combining Lactobacillus plantarum L-137 with a known additive for medicines etc.

The composition of the present invention may be formulated into a quasi-drug or a cosmetic product. Examples of the cosmetic product include cosmetic forms, such as cleaning products such as body wash, hand wash and face wash; skin-care cosmetic products such as facial toner, milky lotion and cream; make-up cosmetic products such as foundation, under makeup base and face powder; etc.

When the composition of the present invention is formulated into the form of a food or drink product, a feed, a medicament, a quasi-drug or a cosmetic product, these formulations or an package insert or a package box thereof may have one or more indications based on the effects of the composition of the present invention, and said one or more indications are selected from the following:

(1) an indication that the formulation promotes fibroblast proliferation,
(2) an indication that the formulation promotes the expression of hyaluronic acid synthase genes, such as HAS1 and HAS2,
(3) an indication that the formulation promotes hyaluronicacid production,
(4) an indication that the formulation has an effect of preventing, ameliorating or treating skin diseases, for example, atopic dermatitis, psoriasis, etc., and
(5) an indication that the formulation has an effect of preventing, ameliorating or treating arthritis, for example, knee pain, cartilage disorder, knee osteoarthritis, cartilage defect, cartilage damage, meniscus injury, etc.

Skin diseases and arthritis can be prevented, ameliorated or treated by promoting fibroblast proliferation and/or promoting hyaluronic acid synthase gene expression.

The composition of the present invention preferably can exhibit one or more effects selected from the following:

(1) an effect of promoting fibroblast proliferation,
(2) an effect of promoting the expression of hyaluronic acid synthase genes, such as HAS1 and HAS2,
(3) an effect of promoting hyaluronic acid production,
(4) an effect of preventing, ameliorating or treating skin diseases, for example, atopic dermatitis, psoriasis, etc., and
(5) an effect of preventing, ameliorating or treating arthritis, for example, knee pain, cartilage disorder, knee osteoarthritis, cartilage defect, cartilage damage, meniscus injury, etc.

In an example, for determining the presence or absence of an effect of promoting fibroblast proliferation, when a sample more effectively promotes fibroblast proliferation than a control with no addition of the sample, the sample can be determined to have an effect of promoting fibroblast proliferation.

In another example, for determining the presence or absence of an effect of promoting hyaluronic acid synthase gene expression, when a sample more effectively promotes hyaluronic acid synthase gene expression than a control with no addition of the sample, the sample can be determined to have an effect of promoting hyaluronic acid synthase gene expression.

In another example, for determining the presence or absence of an effect of promoting hyaluronic acid production, when a sample more effectively promotes hyaluronic acid production than a control with no addition of the sample, the sample can be determined to have an effect of promoting hyaluronic acid production.

In another example, for determining the presence or absence of an effect of ameliorating or treating a skin disease, when a significant difference is observed in the test item “itching” etc. in a survey between before and after the administration of a sample, the sample can be determined to have an effect of ameliorating or treating a skin disease.

In another example, for determining the presence or absence of an effect of ameliorating or treating arthritis, when a significant difference is observed in a score measured by WOMAC (Western Ontario and McMaster Universities Osteoarthritis Index) assessment, SF-36 (MOS 36-Item Short-Form Health Survey), JKOM (Japanese Knee Osteoarthritis Measure) assessment, etc. between before and after the administration of a sample, the sample can be determined to have an effect of ameliorating or treating arthritis.

The present invention provides a method for producing a composition comprising hyaluronic acid, the method comprising allowing (1) (a) Lactobacillus plantarum L-137 and (b) an immune cell or a substance produced by an immune cell to contact with (2) fibroblasts.

The fibroblasts may be those found in the body of a mammal etc. such as a human, or may be those outside of the body of a mammal etc., but are preferably those found in the body of a mammal etc. The contact of the ingredient (1) with the ingredient (2) may be achieved by oral ingestion of the ingredient (1) or application of the ingredient (1) on the skin, or by adding the ingredient (1) to culture medium containing the ingredient (2).

The present invention provides a hyaluronic acid-containing composition produced by the production method as described above (hereinafter, also called the hyaluronic acid-containing composition of the present invention).

The hyaluronic acid-containing composition of the present invention may contain the above ingredient (1), the above ingredient (2), or any ingredient known in the field of medicines, pharmaceuticals, food products, etc.

The hyaluronic acid-containing composition of the present invention preferably exhibits one or more effects selected from the following: an effect of preventing, ameliorating or treating skin diseases, for example, atopic dermatitis, psoriasis, etc.; and an effect of preventing, ameliorating or treating arthritis, for example, knee pain, cartilage disorder, knee osteoarthritis, cartilage defect, cartilage damage, meniscus injury, etc.

The hyaluronic acid-containing composition of the present invention may be a food or drink product, a cosmetic product, a quasi-drug, etc., but is preferably a food or drink product. When the hyaluronic acid-containing composition of the present invention is a food or drink product, specific examples thereof include food additives or the so-called dietary supplements in the form of a tablet, granules, a powder, an energy drink, etc.

### EXAMPLES

The present invention will be described more specifically with reference to the following Examples and experiments, but the present invention is not limited thereto.

### 1. Assessment of Effect of Promoting the Gene Expression of HAS1 and HAS2

### (a) Preparation of Samples (1)

### Example 1

BALB/c mouse spleen immune cells at 2.5×10⁶ cells/ml were cultured in RPMI 1640 medium (Life Technologies) supplemented with FBS (Hyclone) at 10% by mass and containing 500 ng/ml heat-killed bacterial cells of Lactobacillus plantarum L-137 (HK L-137) at 37° C. for 48 hours. The culture medium was recovered to obtain the culture supernatant of the spleen cells. Mouse fibroblasts (BALB/3T3 clone A31) were cultured in D-MEM medium (Sigma) supplemented with FBS (Hyclone) at 2% by mass and containing the culture supernatant of the spleen cells at 25% by mass at 37° C. for 3 hours.

### Comparative Example 1

Culture supernatant of spleen cells was prepared by the same procedures as in the above Example except that culture medium with no addition of HK L-137 was used instead of RPMI 1640 medium (Life Technologies) supplemented with FBS (Hyclone) at 10% by mass and containing 500 ng/ml HK L-137. Mouse fibroblasts (BALB/3T3 clone A31) were cultured in D-MEM medium (Sigma) supplemented with FBS (Hyclone) at 2% by mass and containing the culture supernatant of the spleen cells at 25% by mass at 37° C. for 3 hours.

### Negative Control 1

Mouse fibroblasts (BALB/3T3 clone A31) were cultured in D-MEM medium (Sigma) supplemented with FBS (Hyclone) at 2% by mass at 37° C. for 3 hours.

### Positive Control 1

Mouse fibroblasts (BALB/3T3 clone A31) were cultured in D-MEM medium (Sigma) supplemented with FBS (Hyclone) at 21 by mass and containing 10 ng/ml epidermal growth factor (EGF) at 37° C. for 3 hours.

### (b) Experiments

RNA was extracted from the cultured cells using a kit (RNeasy Mini Kit, Qiagen). The relative mRNA expression levels of the HAS1 and HAS2 genes in total RNA were quantified by RT-PCR. The mRNA expression levels of HAS1 and HAS2 were calculated as a percentage normalized to the mRNA expression level of the internal standard GAPDH. The primer sequences used for the quantification are shown in Table 1.

TABLE 1

| | | | SEQ |
|---|---|---|---|
| | | | ID |
| Gene | Direction | Primer sequence | NO: |
| HAS1 | Forward | 5′-CTATGCTACCAAGTATACCTCG-3′ | 1 |
| HAS1 | Reverse | 5′-TCTCGGAAGTAAGATTTGGAC-3′ | 2 |
| HAS2 | Forward | 5′-CGGTCGTCTCAAATTCATCTG-3′ | 3 |
| HAS2 | Reverse | 5′-ACAATGCATCTTGTTCAGCTC-3′ | 4 |
| GAPDH | Forward | 5′-AATGTGTCCGTCGTGGATCTGA-3′ | 5 |
| GAPDH | Reverse | 5′-AGTGTAGCCCAAGATGCCCTTC-3′ | 6 |

### HAS1

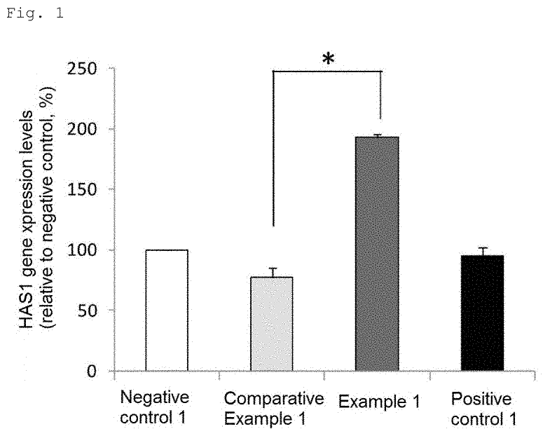

The results are shown in FIG. 1. The gene expression level of Comparative Example 1 was 77% and the gene expression level of Example 1 was 193% as compared with that of Negative control 1 taken as 100%. The HAS1 gene expression level was significantly higher in Example 1 than in Comparative Example 1.

### HAS2

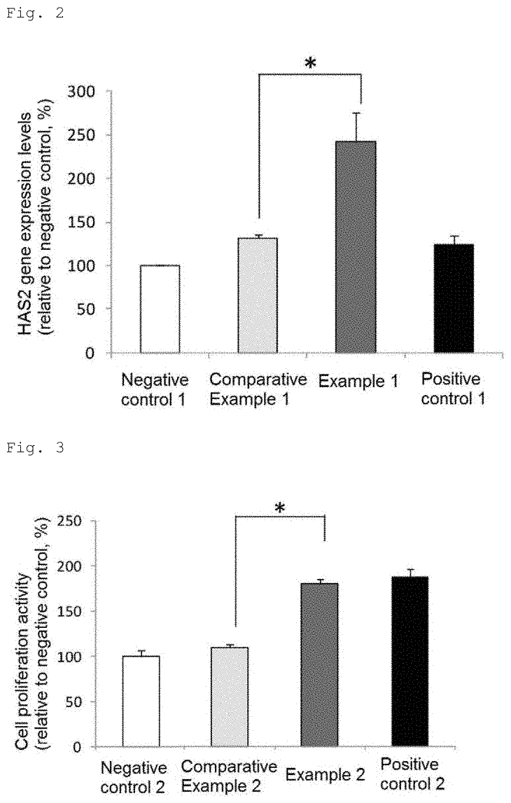

The results are shown in FIG. 2. The gene expression level of Comparative Example 1 was 132% and the gene expression level of Example 1 was 242% as compared with that of Negative control 1 taken as 100%. The HAS2 gene expression level was significantly higher in Example 1 than in Comparative Example 1.

### 2. Assessment of Effect of Activating Fibroblasts

### (a) Preparation of Samples (2)

The samples of Example 2, Comparative Example 2, Negative control 2 and Positive control 2 were prepared by the same procedures as in the above Preparation of samples (1) except that the duration of culture of the mouse fibroblasts (BALB/3T3 clone A31) was changed from 3 hours to 72 hours.

### (b) Experiments

The proliferation (metabolic) activity of the cells was determined by the WST-1 method. Specifically, a solution containing 500 μM WST-1 (WAKO) and 20 μM 1-Methoxy PMS (WAKO) was added to the culture medium prepared in the above (a) to a concentration of 5% by mass. The absorbance of the culture medium at 450 nm was measured immediately after the addition and after culture at 37° C. for 2 hours. An increase in the absorbance was determined as an indicator of the proliferation activity of the cells.

The results are shown in FIG. 3. The proliferation activity of fibroblasts in Comparative Example 2 was 110% and the proliferation activity of fibroblasts in Example 2 was 180% as compared with that in Negative control taken as 100%. The proliferation activity of fibroblasts was significantly higher in Example 2 than in Comparative Example 2.

### 3. Assessment of Effect of Promoting Hyaluronic Acid Synthesis

### (a) Samples

The above samples of Example 2, Comparative Example 2, Negative control 2 and Positive control 2 were used.

### (b) Experiments

The hyaluronic acid levels in the recovered culture medium were measured with an ELISA kit (BTP-96200, Biotech Trading Partners).

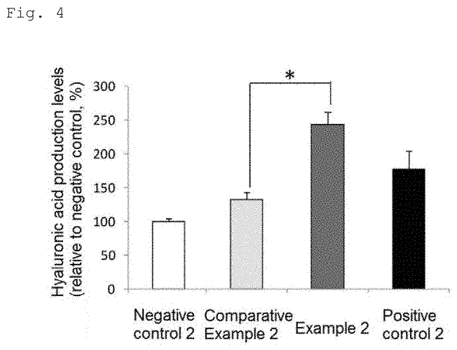

The results are shown in FIG. 4. The production level of hyaluronic acid in Comparative Example 2 was 133% and the production level of hyaluronic acid in Example 2 was 244% as compared with that in Negative control taken as 100%. The production level of hyaluronic acid was significantly higher in Example 2 than in Comparative Example 2.

These results indicate that Lactobacillus plantarum L-137 promotes the expression of hyaluronic acid synthase genes in fibroblasts and/or promotes fibroblast proliferation and hyaluronic acid production.

### INDUSTRIAL APPLICABILITY

The composition of the present invention is useful as a food or drink product, a medicament, a quasi-drug, a cosmetic product, a feed or the like for promoting hyaluronic acid production.

1.-4. (canceled)

5. A method for producing a composition comprising hyaluronic acid, the method comprising allowing (1) (a) Lactobacillus plantarum L-137 and (b) an immune cell or a substance produced by an immune cell to contact with (2) fibroblasts.

6.-8. (canceled)

9. A method for promoting fibroblast proliferation and/or hyaluronic acid synthase gene expression, the method comprising administering an effective amount of Lactobacillus plantarum L-137 to a subject.

10.-12. (canceled)

13. The method according to claim 9, wherein the Lactobacillus plantarum L-137 promotes hyaluronic acid production.

14. The method according to claim 9, wherein the Lactobacillus plantarum L-137 prevents, ameliorates or treats a skin disease or arthritis.

15. The method according to claim 9, wherein the Lactobacillus plantarum L-137 is contained in a food or drink product.

16. The method according to claim 15, wherein the food or drink product is a food additive or a dietary supplement.


SEQUENCE LISTING (6 sequences)

SEQ ID NO: 1 - DNA, 22 bp, Artificial Sequence
  DNA primer
ctatgctaccaagtatacctcg

SEQ ID NO: 2 - DNA, 21 bp, Artificial Sequence
  DNA primer
tctcggaagtaagatttggac

SEQ ID NO: 3 - DNA, 21 bp, Artificial Sequence
  DNA Primer
cggtcgtctcaaattcatctg

SEQ ID NO: 4 - DNA, 21 bp, Artificial Sequence
  DNA primer
acaatgcatcttgttcagctc

SEQ ID NO: 5 - DNA, 22 bp, Artificial Sequence
  DNA primer
aatgtgtccgtcgtggatctga

SEQ ID NO: 6 - DNA, 22 bp, Artificial Sequence
  DNA primer
agtgtagcccaagatgcccttc